(19) United States
(12) Patent Application Publication
(10) Pub. No.: US 2004/0236430 A1
(43) Pub. Date: Nov. 25, 2004

(54) Hip endoprosthesis

(76) Inventors: Rudolf Koch, Oberdorf (CH); Henning Sturz, Giessen (DE)

Correspondence Address: Kaplan & Gilman, 900 Route 9 North, Woodbridge, NJ, 07095, US

(21) Appl. No.: 10/480,184

(22) PCT Filed: Jun. 13, 2001
(86) PCT No.: PCT/IB01/01026

(51) Int. Cl. A61F 2/36

(52) U.S. Cl. 623/23.31; 623/23.35; 623/23.5

(57) ABSTRACT

The invention relates to a cement-free hip endoprosthesis for the femur (1), comprising a shaft (2) which can be inserted into the femur (1) and has a curvature, and comprising at least approximately parallel or concentric grooves (3) or ribs (4) along the shaft surface, the proximal end (5) of the shaft (2) being formed without a collar, and the groove base in the proximal region (6) running out in a shoulder-shape for at least a part of the groove (3b), resulting in a good seat in combination with a short design length.

The invention relates to a cement-free hip endoprosthesis for the proximal femur.

Such prostheses have been disclosed in a very wide range of forms. Some consist of a shaft which can be inserted into the femur and has a curvature and at least approximately parallel grooves or ribs along the shaft surface.

Such a known design has been disclosed, for example, by Link® under the name C.F.P.® hip prosthesis shaft (cf. DE-U1-29705500 and WO-A-98/42279). In this known design, the following have been recognized as being disadvantageous by the inventor: a collar-like neck support which is supported on the resectioned neck of the femur and is thus intended to contribute to good vertical pressure induction is formed at the proximal end. However, the Applicant observed that, as a result of overloading, such neck supports can very rapidly lead to bone degradation in the proximal region of the neck of the femur or to micromovements and to loosening of the distal part of the shaft. Moreover, the Applicant observed that, owing to the length of the known shaft, which extends substantially into the region of the diaphysis, the intervention in the femur which is required during subsequent replacement of the prosthesis is considerable and the prospect for repeated replacement of the prosthesis, as would be expected especially in the case of young patients, is poor. Furthermore—owing to axial displacement limitation due to mechanical overdetermination—the collar-like support prevents optimal conical wedging of the shaft in a pressfit manner in the surgically produced cavity of the femur.

As early as 1949, bone joint prostheses (femural head prostheses) had been considered (DE-C-837294), which prostheses had ribs and were relatively short. However, these prostheses too were based on the supporting effect on the resectioned neck of the femur, by making the spherical head flat on its distal side and providing it with ribs so that it performed a major part of the axial supporting on the neck of the femur. These known prostheses were straight and remained in the patient for only a short time, so that this prosthesis type was discontinued.

Another type of particularly short hip endoprostheses was published in U.S. Pat. No. 6,120,544 and is in the form of a sleeve and has, instead of ribs, a three-dimensional spatial lattice structure which is said to provide particularly good anchoring in the bone. Unfortunately, however, it is precisely the surface structure chosen in the case of this known prosthesis that leads to considerable problems during subsequent removal of the prosthesis, since the bone can grow to a very considerable extent into these surface structures. A further disadvantage recognized in the case of this prosthesis was that it ensures insufficient force induction in the longitudinal direction of the femur, which moreover was also a problem in the case of the above-mentioned design from 1949. The patients are exposed to the danger of suffering an orthopaedic disadvantage due to offset reduction if, on deep positioning of this known prosthesis, the shaft tilts into a valgus position. Furthermore, bone overloads may occur in the region of the lateral support and may lead to atrophy or even to perforation of the bone and hence to an undesired varus position of the implant.

As in the above-mentioned prostheses, in this one, too, a collar-like flange supported on the neck of the femur is provided, which may lead to the stated problems.

Sulzer AG has launched on the market, under the name CLS®, a hip endoprosthesis which dispenses with collar-like neck supports and has, parallel to the shaft, longitudinal ribs which are continuous at the proximal end. Designs comparable therewith are disclosed, for example, in U.S. Pat. No. 4,704,128, in EP-B1-141022 and in US-B1-6168632. These designs have, as a disadvantage, a large shaft length and a lack of support in the vertical direction. Another disadvantage of this design is that it is straight and thus does not fulfil the physiognomic condition of the basically curved bone structures in the femur.

The force induction from the prosthesis to the bone is therefore not optimum physiologically. The large length entails the above-mentioned disadvantages.

Variants comparable with CLS® are described in the following documents. They are all straight and relatively long and, in the inserted state, extend into the region of the diaphysis: EP-A2-494040; EP-A1-669116; EP-A1-821923 and FR-A1-2602672, the latter also having a collar-like support element.

Sulzer AG has also launched on the market, under the name Lamella® (cf. for example: EP-B1-222236; EP-B1-378044), another prosthesis in which relatively sharp-edged and sheet-like broad lamellae are said to permit good anchoring with the bone. However, these designs showed that the deep groove bases formed between the lamellae frequently cannot be reached by bone material, but that, instead of stable intergrowth, rather an accumulation of connective tissue forms in this region and can make only a small contribution to the force transmission. For this reason, this previously considered technology has not become established.

In the area of cementable endoprostheses, which is not considered by the invention and—from the technical point of view—has developed into a separate area of prosthetics, curved prostheses have been published, for example in U.S. Pat. No. 3,874,003. The transmission of the forces takes place not directly from the prosthesis but indirectly by the cement which, however, may be subject to disadvantageous continuous degradation as a result of ageing. The design according to US-A moreover provides a support collar for the neck of the femur.

It is the object of the invention to avoid the stated disadvantages and to provide an endoprosthesis which places as little load as possible on the patient, which nevertheless ensures optimum stability and force transmission and which requires as little removal as possible of bone substance in the event of revisions.

This object is achieved if the proximal end of the shaft is formed without a collar and if the groove base runs out in a shoulder shape in the proximal end. The choice of a curved prosthesis results in optimum force induction, a simple surgical technique and a good seat. In order to counteract lateral support overloading, as described above, the shaft remains fully wedge-shaped on its broad side and deliberately does not have the medial step which is described in U.S. Pat. No. 6,120,544 and which may prevent the advantageous annular stresses in the bone tube. The grooves or the ribs formed by them have the effects, known per se, of good prevention of rotation and tilting of the shaft. However, the shoulders used for the first time in this combination at the proximal groove end serve to ensure the function of absorbing axial forces without (over)loading the bone in parts to such an extent that bone degradation might occur.

According to a particular embodiment of the endoprosthesis according to the invention, the depth of the grooves is 0.5-3.5 mm. It has been found that grooves which are not too deep are preferred for anchoring with the bone, in order to avoid the above-mentioned problems with insufficient integration of bone material in grooves which are too deep.

It has been found to be advantageous if the depth of the grooves varies along their distance since this permits particularly good intergrowth in at least some regions. Moreover, a certain conical support effect can be achieved which also has a positive effect on the axial force transmission and additionally wedges the shaft.

The width of the grooves is advantageously 1.5-6.5 mm, the groove width along its distance preferably varying—preferably continuously. The ratio of the width of the grooves to the width of the ribs in each of a plurality of cross-sections perpendicular to the axis of extension is preferably about 4-12, particularly preferably 5-10, in particular 6-8.

In one embodiment, an endoprosthesis according to the invention has at least one first group of grooves having a length of about 80-95% of the shaft length and optionally at least one second group of grooves having a length of about 60-75% of the shaft length.

As already mentioned, the length of the shaft plays an important role for bone conservation for subsequent revision work, and for radial force transmission between prosthesis and bone. The special formation according to the invention therefore provides for the extension of the shaft in the implanted state to the intertrochanteric region of the femur so that the length of the shaft in the implanted state is limited to the region of the metaphysis.

In order to make the endoprosthesis as short as absolutely necessary, the structural length of the shaft is therefore preferably 15-30% of the length of the femur for which it is intended.

Particular further developments and variants thereof are described or protected in the further Patent Claims.

A symmetrical formation is preferred in that it can then be used for both sides of a patient and also simplifies production and logistics.

As already mentioned for the grooves, the shaft—as known per se—is preferably conical over at least 90% of its longitudinal dimension, the conicity preferably being 2-8°.

The prosthesis according to the invention can, as in the case of conventional prostheses, also be improved on its surface so that optimum bone intergrowth is possible. Depending on requirements, the following measures are provided by a particular embodiment of the invention:

The surface roughness acquires an Ra value of about 3-5 μm, for example by corundum blasting or coarse grinding.

The shaft is provided over its total length with a coating having, in particular, an open-pore structure. This also results in a significant difference compared with the known endoprostheses, which generally have a polished smooth section in the distal region.

Biomimetic materials, hydroxyapatite, porous active material substrate layers and the like help either to improve the initial growth of the bone or to introduce certain active substances or medicaments.

Good bonding results are obtained if, according to a further development of the invention, the shaft is at least partly coated with or composed of an open-cell metal foam or metallized carbon foam, as was disclosed, for example, in U.S. Pat. No. 5,282,861 by Kaplan, titanium or tantalum or an alloy with at least one of the two metals preferably being provided as the metal.

If the prostheses are produced by the forging or hot-pressing process, they are distinguished by particular strength and surface stability, a particularly simple and advantageous process being a forging or hot-pressing process for net-shape blank, so that the shaft need not be subsequently machined.

If all sidewalls of the grooves are parallel to one another or deviate slightly from parallelism for production-related reasons (shaping-related slope of the groove walls), the hot-pressing process has been optimized, the fact that the grooves are not completely identically formed being an additional effect, which is surprisingly advantageous for the intergrowth of the bone—possible due to additional compression.

In the preferred embodiments, the ribs are narrower than the grooves bounded by them, so that the bone has sufficient space for growing into the grooves and can penetrate there to the groove base without complications being expected as a result during subsequent removal for revision purposes.

According to a particularly preferred embodiment, the transitions between the ribs and the grooves are provided with radii of at least 0.6 mm, with the result that the partial loading of the intergrown bone is reduced compared with polygonal or projecting or recessed corners.

The invention is furthermore explained by way of example with reference to drawings. The figures are described in relation to one another. Identical elements carry identical reference numerals. Elements performing similar functions but being differently formed carry identical reference numerals with different indices. The description of the figures, list of reference numerals and patent claims supplement one another in the context of the disclosure of the invention.

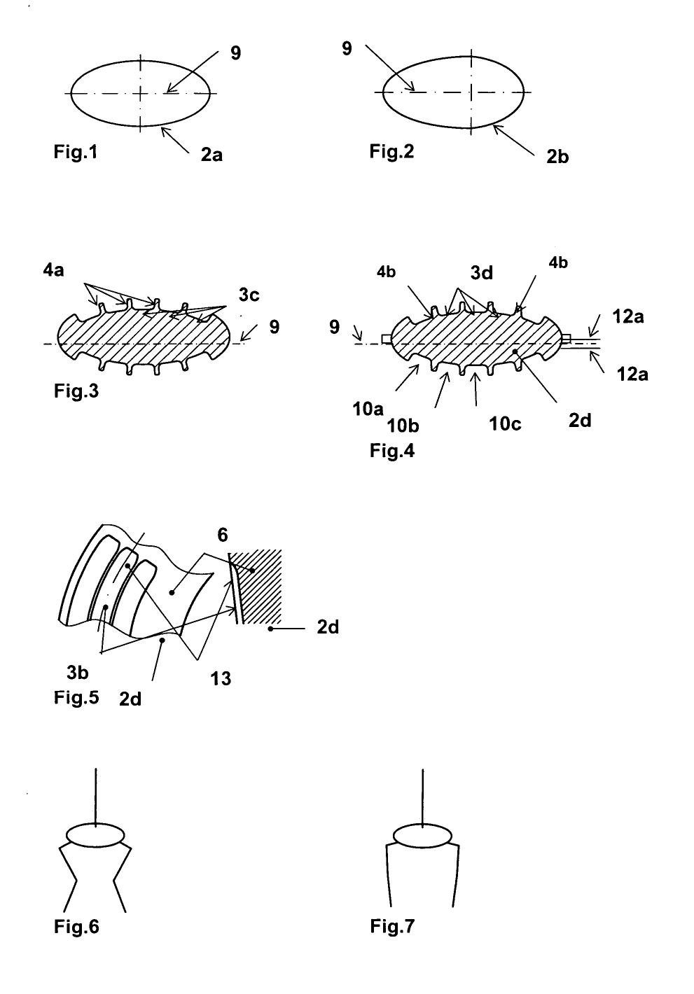

FIG. 1 shows the schematic section through a preferred point-symmetrical or elliptical shaft cross-section;

FIG. 2 shows the schematic section through a linearly symmetrical or oval shaft cross-section;

FIG. 3 shows the schematic section through a shaft according to FIG. 1 having ribs according to the invention;

FIG. 4 shows the schematic section through a variant of the shaft according to FIG. 3 having modified ribs;

FIG. 5 shows a cut-out from the shoulder region of a shaft according to the invention, as a view and as a section;

FIG. 6 shows a schematic valgus view;

FIG. 7 shows a schematic varus view;

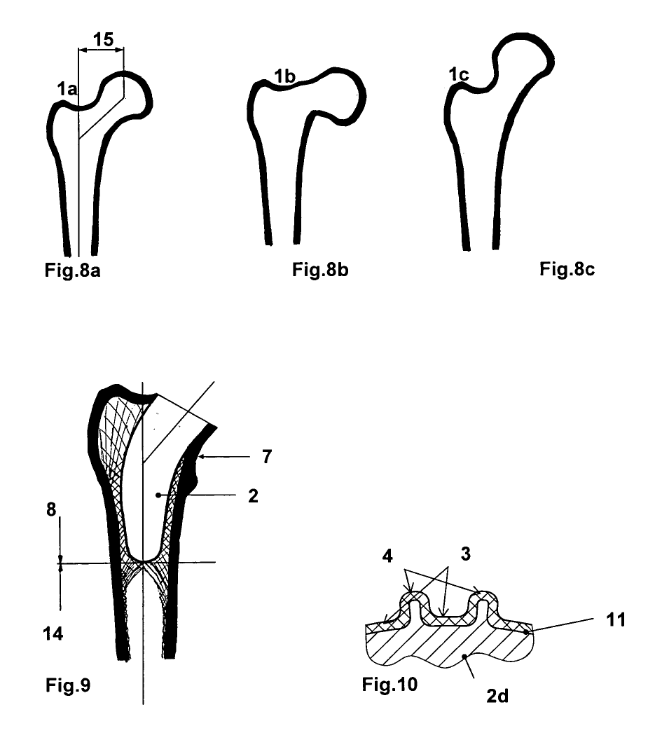

FIG. 8a schematically shows a normal femur bone;

FIG. 8b schematically shows a femur bone tilted into the varus position;

FIG. 8c schematically shows a femur bone tilted into the valgus position;

FIG. 9 schematically shows, in section, a femur bone prepared for the use of an endoprosthesis according to the invention;

FIG. 10 shows a preferred embodiment of the transitions between ribs and grooves in section, with a metal foam coating;

FIG. 11 shows the view of the broad side of an endoprosthesis according to the invention;

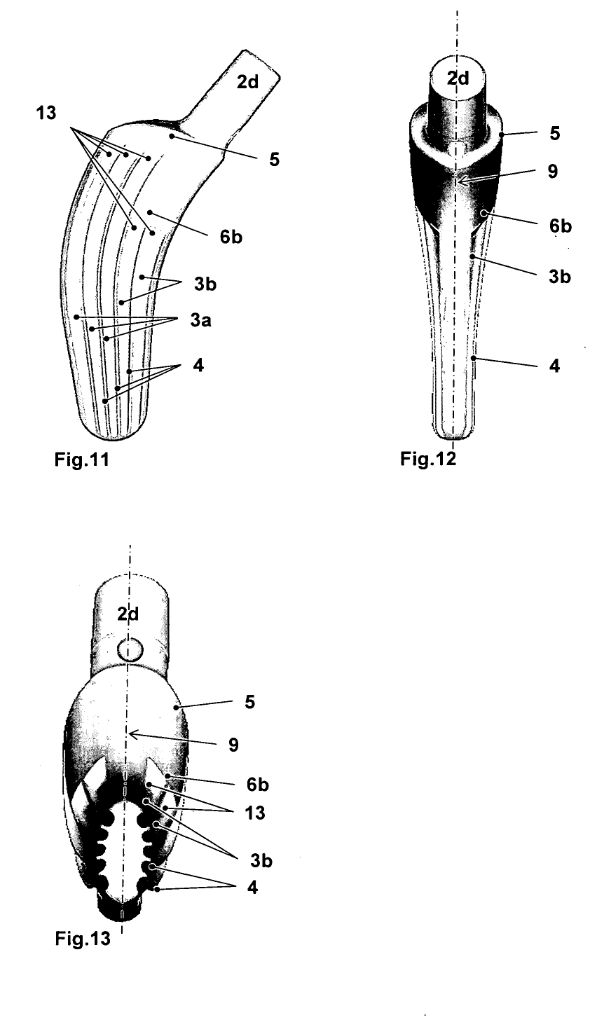

FIG. 12 shows the view of the narrow side of the endoprosthesis according to FIG. 11 and

FIG. 13 shows the distal view of the endoprosthesis according to FIG. 11.

Particularly with regard to the universal applicability (right/left applicability) and possibility of simplified production, a preferred embodiment, according to the invention, of a shaft 2a, 2d is provided with a point-symmetrical elliptical cross-section and is curved only in a plane 9 (FIGS. 1, 3, 4, 11-13). As an alternative, a linearly symmetrical cross-section of a shaft 2b according to FIG. 2 is possible. The shaft 2b, too, can be used on the right and left in only the embodiment curved in the plane 5.

Grooves 3c according to the invention and according to the embodiment in FIG. 3 are approximately radial relative to the centre of the elliptical cross-section. The ribs 4a formed between the grooves 3c thus project in an approximately star-shaped manner. In comparison, the shaft variant according to FIG. 4 exhibits grooves 3d having approximately parallel sidewalls 10a-10d, i.e. in this design the ribs 4b formed between the grooves 3d do not project in a star-shaped manner. 12a or 12b indicate a drop-forging process which, owing to the specially formed ribs 4b and grooves 3d, is capable of producing and shaping the shaft 2d according to FIG. 4 in one operation (net-shape hot pressing).

The grooves 3—as shown in FIG. 5 and at least in part—running out in a shoulder-like manner 13 in the proximal region 5 of the shaft 2d are essential to the invention. This formation permits good axial support without leading to partial overloads and hence to bone shrinkage in the case of the femur bone.

FIG. 9 schematically shows the femur 1 with already resectioned neck of the femur and indicated shaft 2 of the endoprosthesis according to the invention. The shaft 2 is limited with its longitudinal dimension—in the preferred embodiments—substantially to the region of the metaphysis 8 or to the intertrochanteric region 7, and it is for this reason that there are only minimal problems during subsequent revision work and as much substance of the bone as possible is retained. Owing to the curved formation of the shaft 2, the forces are however optimally guided into the femur in spite of the short length. Furthermore, rotation is optimally prevented owing to the ribs and the elliptical cross-section.

The cut-out according to FIG. 10 shows that, in the preferred embodiments, the transitions between grooves 3 and ribs 4 are concave or convex or have a radius which keeps partial loads of the bone small under complete loading of the shaft 2d. A transition 11 comprising metal foam, which permits improved bone growth, is shown schematically.

FIG. 8a shows a normal state of the femur 1a, while FIG. 8b shows a femur in the varus position and 8c a femur in the valgus position. The offset 15 and the position of the femoral head permit a normal leg posture for a patient. If, in the case of an inadequate conventional endoprosthesis, lateral lowering of the femoral head or of the shaft of the prosthesis occurs, the leg develops a varus posture. If, on the other hand, owing to an axial lowering of the shaft, the femoral head or the shaft of the prosthesis tilts proximally, this leads to a valgus posture of the leg. Owing to the shaft formation according to the invention, the states according to 8b and 8c are very substantially avoided.

FIG. 11-13 show an optimum endoprosthesis as can universally be used. In addition to the above-mentioned embodiments, different grooves 3a and 3b are evident as a special feature, the former extending over virtually the total shaft length whereas the latter 3b begin further in the distal direction. In this example shown—and preferably—all grooves have axially supporting shoulders 13.

### List of Reference Numerals

1 Femur

2 Shaft

3 Groove

4 Rib

5 Upper (proximal) end of the shaft

6 Upper (proximal) end of the groove

7 Intertrochanteric region

8 Region of the metaphysis

9 Plane

10 Sidewall of a groove 3

11 Metal foam (or porous metal structure), e.g.: according to U.S. Pat. No. 5,282,861-Kaplan

12 Drop forging process

13 Shoulder-shaped

14 Region of the diaphysis

15 Offset

1. A cement-free hip endoprosthesis for a femur, comprising:
a shaft which can be inserted into the femur and which has a curvature, and
at least approximately parallel or concentric grooves or ribs along the shaft surface and distributed over the total circumference of the shaft,
wherein the curvature of the shaft runs along a continuously curved body axis, a proximal end of the shaft is formed without a collar, and a depth of the grooves in the proximal region is reduced for at least a part of the grooves on the shaft.

2. The endoprosthesis according to claim 1, wherein a depth of the grooves is 0.5-3.5 mm, and/or preferably varies along their longitudinal dimension.

3. The endoprosthesis according to claim 1, wherein a width of the grooves is about 1.5-6.5 mm, and/or the groove width varies—preferably continuously—along their longitudinal dimension.

4. The endoprosthesis according to claim 1, wherein a ratio of a width of the grooves to a width of the ribs in each of a plurality of cross-sections perpendicular to an axis of extension is at least one of: (i) about 4-12, (ii) 5-10, and (iii) 6-8.

5. The endoprosthesis according to claim 1, wherein at least one first group of grooves extends over a length of about 80-95% of a shaft length, and/or at least one second group of the grooves extends over a length of about 60-75% of the shaft length.

6. The endoprosthesis according to claim 1, wherein the shaft has a design length of about 15-30% of a length of the femur for which it is intended.

7. The endoprosthesis according to claim 1, wherein an extension of the shaft in an implanted state is limited to an intertrochanteric region of the femur, or that a length of the shaft in an implanted state is limited to a region of a metaphysis.

8. The endoprosthesis according to claim 1, wherein the shaft is curved only in a plane and is formed preferably symmetrically with respect to the plane.

9. The endoprosthesis according to claim 1, wherein the shaft has a point-symmetrical formation, in particular an elliptical one, at least in a plurality of cross-sections—perpendicular to its axis of extension.

10. The endoprosthesis according to claim 1, wherein the shaft is conical over at least about 90% of its longitudinal dimension, the conicity preferably being about 2-8°.

11. The endoprosthesis according to claim 1, wherein a surface roughness of the shaft corresponds to an Ra value of about 3-5 μm.

12. The endoprosthesis according to claim 1, wherein the shaft has a coating with an open-pore structure and/or is roughened over its total length.

13. The endoprosthesis according claim 1, wherein the shaft is coated with at least one of the following materials: porous metal layer, biomimetic materials, hydroxyapatite, and porous active substance substrate layers.

14. The endoprosthesis according to claim 1, wherein the shaft is coated at least partly with an open-cell metal foam or metallized carbon foam or is composed of such a foam, and the metal is titanium, tantalum or an alloy comprising at least one of the two metals.

15. The endoprosthesis according to claim 1, wherein the endoprosthesis is at least one of: (i) composed of titanium or a titanium alloy, and (ii) is produced by a drop forging or hot pressing process.

16. The endoprosthesis according to claim 1, wherein the endoprosthesis is produced by a drop forging or hot pressing process as a net-shape blank which does not have to be subsequently machined.

17. The endoprosthesis according to claim 1, wherein all sidewalls of the grooves are parallel to one another or deviate slightly from parallelism.

18. The endoprosthesis according to claim 1, wherein at least one of: (i) the ribs are narrower than the grooves bounded by them, and/(ii) transitions between the ribs and the grooves are provided with radii of at least about 0.6 mm.
